(19) United States
(12) Patent Application Publication
(10) Pub. No.: US 2004/0197839 A1
(43) Pub. Date: Oct. 7, 2004

(54) Methods of detecting cancer cells in biological samples

(75) Inventors: Michal Daniely, Ganey Tikava (IL); Tal Kaplan, Ashdod (IL); Eran Kaplan, Rehovot (IL); Avner Freiberger, Rishon Lezion (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: Bioview Ltd.

(21) Appl. No.: 10/771,440

(22) Filed: Feb. 5, 2004

Related U.S. Application Data
(60) Provisional application No. 60/459,992, filed on Apr. 4, 2003.

(51) Int. Cl. G01N 33/53; G01N 33/574; G01N 1/30; G01N 33/48

(52) U.S. Cl. 435/7.23; 435/40.5

(57) ABSTRACT

The invention provides methods of detecting cancerous cells in biological samples using a double staining/dual imaging approach, which can be used to diagnose cancer. More specifically, the present invention provides methods of diagnosing bladder cancer by a simultaneous scanning of cell morphology and FISH signals of cells derived from a urine sample.

This application claims the benefit of priority from U.S. provisional patent application No. 60/459,992, filed Apr. 4, 2003.

### FIELD AND BACKGROUND OF THE INVENTION

The present invention relates to methods of detecting cancer cells in biological samples using a double staining/dual imaging approach, more particularly, embodiments of the present invention relate to a method of detecting transitional cell carcinoma in voided urine samples using dual imaging with consecutive scanning of cell morphology and FISH signals.

Early detection of cancer is the key feature in treating cancer patients. For many types of cancer, such as breast cancer, detection is often possible via physical examination of the cancer tissue. In other types of cancer, such as in leukemia, cancer detection is based on the examination of cancerous cells in blood or bone marrow samples, while in kidney or bladder cancers, the cancerous cells can be detected in voided urine. Thus the identification of cancer cells in biological samples may present an accurate approach for cancer diagnosis.

Typically, biological samples are prepared by fixing the cells onto microscopic slides and staining them using a variety of staining methods (e.g., morphological or cytogenetical stains). Stained specimens are then evaluated for the presence or absence of cancerous or abnormal cells.

For example, cytological staining can detect the presence of transitional cell carcinoma (TCC), a malignant tumor, in a urine sample. In many cases, TCC progress from benign papillomas which protrude from the bladder surface and grow into the bladder lumen. However, at lower grades of this progression, cancerous cells are rare and in some cases appear similar to those seen in other conditions not related to cancer such as inflammation, obstruction or stones. On the other hand, in higher grades, the cancerous cells can be detected more easily as their relative number is increased and they have characteristic appearances such as enlarged nuclei and irregular nuclear borders. Thus, cytological staining detects 78% of grade II tumors and 90% of high-grade lesions. However, the highly curable grade I tumors are virtually undetectable using cytological staining.

Cytological staining methods have several other disadvantages especially in cases where there are no identifiable tumors or pre-cancerous lesions. For example, the detection of lung cancer using sputum samples requires the presence of at least one relatively rare cancer cell in a sputum sample. In these cases, the accuracy of cancer detection is highly dependent on the experience of the pathologist viewing the specimens.

Although often inconclusive, cytological staining methods are the most common methods currently practiced for the detection of cancerous cells in biological samples.

Other staining methods often used for cancer detection include immunohistochemistry and activity stains. These methods are based on the presence or absence of specific antigens or enzymatic activities in cancerous cells. For example, bladder cancer can be detected using several urine markers such as the nuclear matrix protein (NMP-22), the bladder tumor antigen (BTA), and the telomerase which is expressed in 90% of bladder cancers [Orlando, C. et al., (2001). Telomerase in urological malignancy. J. Urol. 166: 666-73]. In general, each of these markers has better sensitivity than cytology alone but is prone to more false-positive findings.

Other methods of detecting cancerous cells utilize the presence of chromosomal aberrations in cancer cells. In particular, the deletion or multiplication of copies of whole chromosomes or chromosomal segments, and higher levels of amplifications of specific regions of the genome are common occurrences in cancer [Smith, et al., (1991). Breast Cancer Res. Treat., 18: Suppl. 1: 5-14; van de Vijer & Nusse (1991). Biochem. Biophys. Acta. 1072: 33-50; Sato, et al., (1990). Cancer. Res., 50: 7184-7189].

Bladder cancer is also associated with chromosomal aberrations. Approximately 60-65% of all TCC tumors are characterized by loss of heterozygosity (LOH) on chromosome 9. Allelic loss of chromosome 9 is considered to be one of the earliest events in the development of bladder cancer and is found exclusively in early-stage, well-differentiated tumors. In contrast, LOH of chromosome 17, especially on the short arm, is noted in about 40% of bladder tumors, and especially in high-grade, high-stage tumors [Orlow, I. Et al., (1995). Deletion of the p16 and p15 genes in human bladder tumors. J. Natl. Cancer. Inst. 87: 1524-1529; Poddighe, P. J. (1996). Loss of chromosome 9 in tissue sections of transitional cell carcinomas as detected by interphase cytogenetics. A comparison with RFLP analysis. Journal of Pathology, 179: 169-176]. Cytogenetic studies reveal frequent gains of a variety of chromosomes, including chromosome 9, 17, 7, 11, 1, 3 and others [Ishiwata, S. et al (2001). Noninvasive detection and prediction of bladder cancer by fluorescence in situ hybridization analysis of exfoliated urothelial cells in voided urine. Urol, 57(4): 811, 2001; Marano, A. et al., (2000). Chromosomal numerical aberrations detected by fluorescence in situ hybridization on bladder washings from patients with bladder cancer. Eur Urol, 37: 358]. Further LOH on chromosomes 3, 4, 5, 6, 8, 11, 13 and 18 are found in tumors that penetrate into the muscularis layers and spread beyond the bladder wall. In addition, loss of chromosomes 2q, 4, 8p, and 11p; gain of chromosome 17; and amplification at 11q12q13 are found in invasive papillary bladder cancer [Obermann, E. C. et al., (2003). Frequent genetic alterations in flat urothelial hyperplasias and concomitant papillary bladder cancer as detected by CGH, LOH, and FISH analyses. J Pathol. 199: 50-7].

Chromosomal aberrations are often detected using cytogenetic methods such as Giemsa-stained chromosomes (G-banding) or fluorescent in situ hybridization (FISH). FISH is considered an advanced approach over cytogenetic and is often used for the detection of bladder cancer.

Typically, biological specimens, stained by any of the methods described hereinabove, are manually evaluated by either a lab technician or a pathologist. Microscopic slides are first viewed under low magnification to locate candidate areas and those areas are then viewed under higher magnification to evaluate the presence of cancerous cells.

Thus, the current methods of assessing biological specimens are time consuming and are prone to diagnostic errors resulting from missing slide areas and misinterpretation of the stained sample.

In addition, since current approaches utilize a single staining method at a time, such approaches increase the chance of either false negative results associated with cytological staining methods or false positive results associated with immunogenic or activity-based staining methods.

There is thus a widely recognized need for, and it would be highly advantageous to have, a method of diagnosing and screening of cancer cells in general, and transitional cell carcinoma of the bladder, in particular, devoid of the above limitations.

### SUMMARY OF THE INVENTION

According to one aspect of the present invention there is provided a method of identifying cancerous cells in a biological sample comprising: (a) staining nucleated cells of the biological sample with at least two stains to thereby obtain stained nucleated cells; and (b) sequentially and/or simultaneously exposing the stained nucleated cells to at least two imaging modes, to thereby identify the cancerous cells in the biological sample.

According to another aspect of the present invention there is provided a method of diagnosing cancer in a subject, the method comprising: (a) obtaining a biological sample from the subject; (b) staining nucleated cells of the biological sample with at least two stains to thereby obtain stained nucleated cells, and; (c) sequentially and/or simultaneously exposing the stained nucleated cells to at least two imaging modes, to thereby determine the presence or absence of cancerous cells within the stained nucleated cells, wherein presence of the cancerous cells is indicative of a positive cancer diagnosis.

According to yet another aspect of the present invention there is provided a method of identifying transitional cell carcinoma cells in a urine sample comprising: (a) staining nucleated cells of the urine sample with at least two stains to thereby obtain stained nucleated cells, and; (b) sequentially and/or simultaneously exposing the stained nucleated cells to at least two imaging modes, to thereby identify the transitional cell carcinoma cells in the urine sample.

According to still another aspect of the present invention there is provided a method of diagnosing bladder cancer in a subject, the method comprising: (a) obtaining a urine sample from the subject; (b) staining nucleated cells of the urine sample with at least two stains to thereby obtain stained nucleated cells, and; (c) sequentially and/or simultaneously exposing the stained nucleated cells to at least two imaging modes, to thereby determine the presence or absence of cancerous cells within the stained nucleated cells, wherein presence of the cancerous cells is indicative of a positive cancer diagnosis.

According to further features in preferred embodiments of the invention described below, each imaging mode of the at least two imaging modes is specific to a stain of the at least two stains.

According to still further features in the described preferred embodiments the cancerous cells are associated with a cancer selected from the group consisting of leukemia, lymphoma, brain cancer, cerebrospinal cancer, bladder cancer, prostate cancer, breast cancer, cervix cancer, uterus cancer, ovarian cancer, kidney cancer, esophagus cancer, lung cancer, colon cancer, and melanoma.

According to still further features in the described preferred embodiments the biological sample is selected from the group consisting of bone marrow cells, lymph nodes cells, peripheral blood, cerebrospinal fluid, urine, effusions, fine needle aspirates and/or peripheral blood scrapings, paraffin embedded tissue, and frozen sections.

According to still further features in the described preferred embodiments the transitional cell carcinoma cells are associated with bladder cancer and/or kidney cancer.

According to still further features in the described preferred embodiments the urine sample is obtained via voided urine or catheterization.

According to still further features in the described preferred embodiments each stain of the at least two stains is independently selected from the group consisting of a morphological stain, an immunological stain, an activity stain, a cytogenetical stain, in situ hybridization stain and a DNA stain.

According to still further features in the described preferred embodiments the morphological stain is selected from the group consisting of May-Grünwald-Giemsa stain, Giemsa stain, Papanicolau stain, Hematoxylin-Eosin stain and DAPI stain.

According to still further features in the described preferred embodiments the immunological stain is selected from the group consisting of fluorescently labeled immunohistochemistry, radiolabeled immunohistochemistry and immunocytochemistry.

According to still further features in the described preferred embodiments the activity stain is selected from the group consisting of cytochemical stain and substrate binding assay stain.

According to still further features in the described preferred embodiments the cytogenetical stain is selected from the group consisting of G-banding stain, R-banding stain, Q-banding, and C-banding.

According to still further features in the described preferred embodiments the in situ hybridization stain is selected from the group consisting of fluorescent in situ hybridization (FISH) stain, radiolabeled in situ hybridization stain, Digoxigenin labeled in situ hybridization stain and biotinylated in situ hybridization stain.

According to still further features in the described preferred embodiments the DNA stain is a DNA-binding fluorescent dye.

According to still further features in the described preferred embodiments a first stain of the at least two stains is a morphological stain and a second stain of the at least two stains is selected from the group consisting of an immunological stain, an activity stain, an in situ hybridization stain, and a DNA stain.

According to still further features in the described preferred embodiments a first stain of the at least two stains is an immunological stain and a second stain of the at least two stains is selected from the group consisting of a morphological stain, an activity stain, an in situ hybridization stain, and a DNA stain.

According to still further features in the described preferred embodiments a first stain of the at least two stains is an activity stain and a second stain of the at least two stains is selected from the group consisting of a morphological stain, an immunological stain, an in situ hybridization stain, and a DNA stain.

According to still further features in the described preferred embodiments a first stain of the at least two stains is a cytogenetical stain and a second stain of the at least two stains is selected from the group consisting of an immunological stain, an in situ hybridization stain, and a DNA stain.

According to still further features in the described preferred embodiments a first stain of the at least two stains is an in situ hybridization stain and a second stain of the at least two stains is a DNA stain.

According to still further features in the described preferred embodiments a first stain of the at least two stains is a DNA stain and a second stain of the at least two stains is an in situ hybridization stain.

The present invention successfully addresses the shortcomings of the presently known configurations by providing methods of detecting cancerous cells in biological samples using at least double staining and dual imaging.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b are photomicrographs of voided urine sample cells illustrating FISH (FIG. 1a) and cytological (FIG. 1b) analyses. Shown is an abnormal epithelial cell exhibiting a high nucleus to cytoplasm (N/C) ratio using May-Grünwald-Giemsa stain (FIG. 1b, cell marked with square brackets, magnification×20), a large and irregular nucleus using DAPI stain (FIG. 1a, blue counterstain, magnification×63) and abnormal FISH signals with polyploidy of chromosomes 3, 7, and 17 (FIG. 1a, red, green and aqua signals, respectively, magnification×63).

FIGS. 2a-b are photomicrographs of voided urine sample cells illustrating FISH (FIG. 2a) and cytological (FIG. 2b) analyses. Shown is an epithelial cell exhibiting a large and irregular nucleus using DAPI stain (FIG. 2a, blue counterstain, magnification×63), however with normal FISH signals (FIG. 2a, red, green and aqua signals, magnification×63) and normal morphology using May-Grünwald-Giemsa stain (FIG. 2b, cell marked with square brackets, magnification×20).

FIGS. 3a-b are photomicrographs of voided urine sample cells illustrating FISH (FIG. 3a) and cytological (FIG. 3b) analyses. Shown is an apparently normal epithelial cell based on the DAPI stain (FIG. 3a, blue counterstain, magnification×63) and the May-Grünwald-Giemsa stain (FIG. 3b, cell marked with square brackets, magnification×20), however, with abnormal FISH signals showing multiple gains of chromosomes 3, 7 and 17 (FIG. 3a, red, green and aqua signals, respectively, magnification×63).

FIGS. 4a-b are photomicrographs of voided urine sample cells illustrating FISH (FIG. 4a) and May-Grünwald-Giemsa (FIG. 4b) analyses. Shown is an abnormal epithelial cell exhibiting a high N/C ratio and a considerable dark appearance under May-Grünwald-Giemsa stain (FIG. 4b, cell marked with square brackets, magnification×20) and a large nucleus with multiple gains of chromosome 3, 7 and 17 (FIG. 4a, red, green and aqua signals, respectively, magnification×63).

### DESCRIPTION OF THE PREFERRED EMBODIMENTS

The present invention is of methods of detecting cancerous cells in biological samples using a double staining/dual imaging approach, which can be used to diagnose cancer. Specifically, the present invention can be used to diagnose bladder cancer by a simultaneous scanning of cell morphology and FISH signals of cells derived from a urine sample.

The principles and operation of the methods of detecting cancerous cells in biological samples and of diagnosing cancers according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Early detection of cancer is the key step in curing cancer. In many types of cancer such as breast cancer, cervical cancer and bladder cancer, early diagnosis can significantly improve patient's prognosis and survival chances.

However, in many cases, once a cancerous tumor is identified it has already progressed into an invasive form. For example, in bladder cancer, although tumors can be removed by a surgical resection, 50-80% of patients experience a recurrent invasive disease with poor prognosis [Itoku, K. A. et al., Superficial Bladder Cancer. In: “Hematology/Oncology Clinics of North America”, P. W. Kantoff et al., eds., W. B. Saunders Co., Philadelphia, pp. 99-116 (1992)].

Efforts have been made to develop methods for early diagnosis of cancer including the detection of cancerous cells in biological samples. Cancerous cells can be detected in biological samples such as peripheral blood and urine samples by staining the specimens with a variety of stains. The staining methods are designed to differentiate cancerous cells, or pre-cancerous cells from the normal cells present in the specimen. Staining methods include cytological stains which are based on the morphology of the cells, immunohistochemistry and activity stains, which rely on the presence or absence of antigens and enzymatic activities in the cancerous cells, and DNA and chromosome stains which detect the presence of chromosomal abnormalities often associated with cancer.

For a comprehensive detection of cancerous cells in biological specimens all of the abovementioned diagnostic methods should be employed, preferably on the same specimen.

Although advantageous, multiple staining of a single specimen and a simultaneous viewing of at least double staining is not currently practiced for the detection of cancerous cells. In order to stain a single sample with more than one type of stain (e.g.. morphological stain and in situ hybridization stain), cell preparation must be conducted such that a recovered cell sample is highly amenable to more than one staining procedure since a specific set of conditions used for one staining method are usually inappropriate for use in another staining method. In addition, for an accurate diagnosis, the two staining methods should be compatible with dual imaging.

While reducing the present invention to practice, the present inventors have uncovered a method of detecting cancerous cells in biological samples.

As described hereinunder and in Example 2 of the Examples section which follows, the resolution of detection of cancerous cells using the combined staining/dual imaging method of the present invention is substantially higher than that of any known prior art approach and thus the present method substantially improves early cancer detection capabilities.

Thus, according to one aspect of the present invention there is provided a method of identifying cancerous cells in a biological sample. Cancerous cells are cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within the body, or may be a non-tumorigenic cancer cell, such as a leukemia cell. Cancerous cells can be associated with many kinds of cancers including, but not limited to leukemia, lymphoma, brain cancer, cerebrospinal cancer, bladder cancer, prostate cancer, breast cancer, cervix cancer, uterus cancer, ovarian cancer, kidney cancer, esophagus cancer, lung cancer, colon cancer, melanoma, neuroblastoma, and pancreatic cancer.

The method is effected by staining nucleated cells of the biological sample with at least two stains to thereby obtain stained nucleated cells; and sequentially and/or simultaneously exposing the stained nucleated cells to at least two imaging modes, to thereby identify the cancerous cells in the biological sample.

The biological sample utilized by the present invention can include bone marrow cells, lymph nodes cells, peripheral blood cells, cerebrospinal fluid, urine and the like. Such samples can be collected using effusions, fine needle aspirates, peripheral blood scrapings, paraffin embedded tissues, frozen sections and the like.

The biological sample is processed and the nucleated cells of the biological sample are stained with at least two stains and visualized using two imaging modalities. For example, biological samples such as blood, bone marrow aspirates and urine samples are centrifuged in the presence of a morphology preserver, such as the one included in the BioWhite kit (BioView LtD., Rehovot, Israel), to prepare cytospin slides suitable for at least two types of stains. Slides are then subjected to a first stain, such as for example a cytological stain (e.g., May-Grünwald-Giemsa, Giemsa, Papanicolau or Hematoxylin-Eosin) which labels the nuclear and cytoplasmic compartments of the cell and enables the screening of morphological abnormalities typical to cancerous cells. Stained cells are then scanned using an imaging apparatus such as the Bio View Duet™ (Bio View, Rehovot, Israel) using an imaging modality suitable for the first stain. For example, if May-Grünwald-Giemsa stain is employed then a bright field modality is used. Cells with abnormal morphology are identified and their images are captured and saved along with the cell's coordinates. Following cell scanning, slides are prepared to the second stain which is capable of detecting cancer specific markers, such as, chromosomal abnormalities, gain or absence of specific antigens on the cell surface, and/or the presence or absence of specific enzymatic activities. Following the second stain, slides are scanned using a different imaging modality for the presence of abnormal cells. Preferably, the second scan follows the coordinates selected in the first scan, however, other modes of scanning are also suitable. It will be appreciated that a cell is considered as a cancerous cell if it exhibits abnormal findings according to both staining methods.

Following is a non-limiting description of a number of staining procedures and approaches for visualizing such stains, which can be utilized by the present invention.

Morphological Stains

Morphological stains bind non-specifically to cell compartments rendering them visible for microscopic observation. Examples include but are not limited to May-Grünwald-Giemsa stain, Giemsa stain, Papanicolau stain, Hematoxylin-Eosin stain, DAPI stain and the like.

Morphological staining can be effected by simple mixing, diluting and washing laboratory techniques and equipment. Following the application of the appropriate stain, the microscopic slides containing stained cells can be viewed under a microscope equipped with either a bright or a dark field source of light with the appropriate filters according to manufacturer's instructions. For example, May-Grünwald-Giemsa stain, Giemsa stain, Papanicolau stain and/or Hematoxylin-Eosin stain can be viewed using bright field modality. On the other hand, DAPI stain is viewed using a dark field modality with a UV lamp.

Immunological Stains

Immunological staining is based on the binding of labeled antibodies to antigens present on the cells. Examples of immunological staining procedures include but are not limited to, fluorescently labeled immunohistochemistry, radiolabeled immunohistochemistry and immunocytochemistry.

Immunological staining is preferably followed by counterstaining the cells with a dye which binds to non-stained cell compartments. For example, if the labeled antibodies bind to antigens present on the cell cytoplasm, a nuclear stain (e.g., Hematoxylin-Eosin stain) is an appropriate counterstaining.

Antibody labeling can be effected using numerous labeling modes known in the art.

For example, antibodies can be conjugated to a fluorescent dye (e.g. fluorescent immunohistochemistry) in which case visualization is direct using a fluorescent microscope and a dark field image modality.

Antibodies can also be radiolabeled with certain isotopes, in which case bound antibodies are retrieved following the development of a photographic emulsion which results in localized silver grains in cells containing bound antibodies. These silver grains can be further viewed under a microscope using a bright field modality.

Alternatively, antibodies can be conjugated to an enzyme (e.g., horseradish peroxidase (HRP)) in which case, upon binding to a chromogenic substrate specific to the conjugated enzyme, the enzyme catalyzes a reaction in which the chromogenic substrate becomes detectable when viewed under a light or a fluorescent microscope.

Activity Stains

According to this method, a chromogenic substrate is applied on the cells containing an active enzyme. The enzyme catalyzes a reaction in which the substrate is decomposed to produce a chromogenic product visible by a light (e.g., bright field modality) or a fluorescent microscope (e.g., dark field modality). Examples of commonly practiced activity staining procedures include but are not limited to cytochemical stain and substrate binding assays.

Substrate binding assays utilize endogenous substrates in order to activate a chromogenic dye bound to an ectopically introduced enzyme. In this method, once the enzyme binds to its natural substrate on the cell, a conformational change within the enzyme molecule activates the conjugated dye in such a way that a chromogenic product will deposit on the cell. The chromogenic product can be further viewed under a light microscope using bright field modality or under a fluorescent microscope using dark field modality.

Cytogenetical Stains

Cytogenetical stains are useful for karyotyping and identifying major chromosomal aberrations. Conventional banding techniques include G-banding (Giemsa stain), Q-banding (Quinacrine mustard stain), R-banding (reverse-Giemsa), and C-banding (centromere banding). Chromosomes are typically examined by bright-field microscopy after Giemsa staining (G-banding), or by fluorescence microscopy using dark field modality after fluorescence staining (R-banding), to reveal characteristic light and dark bands along their length. Careful comparison of a patient's banding pattern with those of normal chromosomes can reveal abnormalities such as translocations (exchange of genetic material between or within chromosomes), deletions (missing chromosome(s) or fragment(s) thereof), additions, inversions and other defects that cause deformities and genetic diseases.

In situ Hybridization Stains

In situ hybridization is a useful method of detecting major and/or minor chromosomal aberrations. In this method labeled nucleic acid probes are denatured and applied on fixed and denatured cells in either the metaphase or the interphase stages of cell cycle. The attachment of the labeled probes to their genomic counterparts reveals specific signals, which can be detected using a microscope. Examples for in situ hybridization include, but are not limited to fluorescent in situ hybridization (FISH), radiolabeled in situ hybridization, Digoxigenin labeled in situ hybridization and biotinylated in situ hybridization.

Numerous nucleic acid labeling techniques are known in the art. For example, a fluorescent dye can be covalently attached to either the 5′ or 3′ end of a nucleic acid probe. Following hybridization, the labeled probe can be directly retrieved using a fluorescent microscope and a dark field modality.

Alternatively, a nucleic acid probe can be directly labeled with a radioactively labeled nucleotide such as ³⁵S-ATP. In this case the labeled nucleotide can be incorporated to the nucleic acid probe by conventional labeling techniques known to those skilled in the art of molecular biology. Labeling techniques used by the present invention include, but are not limited by, Nick Translation, Random Primed Labeling, End Labeling with a polynucleotide kinase etc. Following hybridization, the labeled nucleic acid probes are retrieved by the development of a photographic emulsion which produces dark silver grains that can be further viewed under a light microscope using bright field modality.

Optionally, a nucleic acid probe can be prepared by incorporating a Digoxigenin (DIG) labeled nucleotide to the nucleic acid probe. Digoxigenin labeled nucleotides are prepared according to the labeling techniques described herein above. Following hybridization, an anti-DIG antibody is applied on the cells. Anti-DIG antibodies can be directly labeled with a fluorescent dye in which case the hybridization signal is viewed under a fluorescent microscope using dark field modality or they can be conjugated to an enzyme (e.g., HRP), in which case upon the addition of a chromogenic substrate will produce a color that can be further viewed under a microscope using bright field or dark field modalities.

The nucleic acid probes of the present invention can be also conjugated to a biotin molecule at the 5′ or 3′ end of the nucleic acid probe. In this case, following hybridization, an avidin or a streptavidin molecule is further applied on the cells. The avidin or streptavidin molecules used by the present invention can be directly labeled with a fluorescent dye or can be conjugated to an enzyme which will further produce a chromogenic product once the appropriate substrate is employed. It will be appreciated that fluorescent avidin or streptavidin molecules are further detected under a fluorescence microscope using a dark field modality. However, if a chromogenic product is to be produced the in situ hybridization stained slides are usually viewed under a light microscope using a bright field modality.

DNA Stains

DNA stains are based on the attachment of fluorescent dyes to DNA molecules in order, for example, to quantitate the amount of DNA present in the cells at a specific time. For example, during replication, the amount of DNA/chromosome per cell is multiplied, i.e., from 2N to 4N chromosomes.

Examples for DNA stains include, but are not limited to 4′,6-diamidino-2-phenylindole (DAPI), Propidium Iodide (PI) and Ethidium bromide which can be viewed under a fluorescence microscope using a dark field modality.

When utilized for single staining single imaging analysis of cells, each of the abovementioned staining method is limited by either false negative results (e.g., morphological and in situ hybridization stains) or false positive results (e.g., immunological and activity stains). The present inventors postulated that multiple staining-multiple imaging of a biological sample could substantially reduce such false positive or false negative results.

Indeed, as is illustrated in Examples 1 and 2 of the Examples section which follows, the present inventors have uncovered that staining nucleated cells with two stains and utilizing two different imaging modalities substantially increases the ability to accurately detect cancerous cells in a biological sample. As is illustrated in Table 1 of Example 2, cytology analysis detected 15 out of 21 confirmed cases of TCC, while dual staining-dual imaging analysis practiced according to the teachings of the present invention detected all 21 confirmed cases of TCC.

Examples of staining-imaging pairs include:

(i) a morphological stain such as a May-Grünwald-Giemsa stain, a Giemsa stain, a Papanicolau stain or a Hematoxylin-Eosin stain which can be visualized via light microscopy and an immunological stain using a fluorescently labeled antibody such as fluorescein conjugated anti-p53 (Pantropic) antibody (OP43F, Calbiochem, San Diego, Calif.) which can be visualized via fluorescent microscopy.

(ii) a morphological stain such as DAPI stain which can be visualized via fluorescent microscopy and an immunological stain using a radiolabelled antibody such as for example, Indium-111 labeled F(ab′)2 fragments of monoclonal antibodies directed against the 17-1A and 19-9 gastrointestinal cancer markers [Watanabe, Y. et al., J. Nuc. Med. (1988), 29: 1436-42] or immunocytochemistry [e.g., monoclonal antibodies for cytokeratins (Vagunda, V. et al., Eur. J. Cancer, 2001, 37:1847-52) or MIB-1 (Lin, O. et al., Am. J. Clin. Pathol., 2003, 120: 209-16)] which can be visualized via light microscopy.

(iii) a morphological stain such as DAPI stain which can be visualized via fluorescent microscopy and an activity stain such as a cytochemical stain (e.g., glucose-6-phosphatase, alkaline phosphatase) and substrate binding assay stain (e.g., using Vector Blue) which can be visualized via light microscopy.

(iv) a morphological stain such as May-Grünwald-Giemsa stain, Giemsa stain, Papanicolau stain, Hematoxylin-Eosin stain which can be visualized via light microscopy and fluorescent in situ hybridization (FISH) stain using for example the UroVysion kit probes (Vysis Inc, Downers Grove, Ill., USA) which can be visualized via fluorescent microscopy.

(v) a morphological stain such as DAPI stain which can be visualized via fluorescent microscopy and an in situ hybridization stain using radiolabeled probes such as ³⁵S-, ³²P-labeled DNA probes, Digoxigenin or biotinylated labeled probes conjugated to either horseradish peroxidase and using substrates such as diaminobenzidine (DAB), tetramethylbenzidine (TMB) or to alkaline phosphatase and using substrates such as APase/fast red which can be visualized via light microscopy.

(vi) a morphological stain such as May-Grünwald-Giemsa stain, Giemsa stain, Papanicolau stain, Hematoxylin-Eosin stain which can be visualized via light microscopy and an activity stain such as a cytochemical stain using for example glutathione-mercury orange complexes (Larrauri, A. et al., J. Histochem. Cytochem. 1987, 35: 271-4) and a substrate binding assay stain which can be visualized via fluorescent microscopy.

(vii) a morphological stain such as May-Grünwald-Giemsa stain, Giemsa stain, Papanicolau stain, Hematoxylin-Eosin stain which can be visualized via light microscopy and a DNA-binding fluorescent dye such as DAPI or Ethidium bromide which can be visualized via fluorescent microscopy.

(viii) an immunological stain using a radiolabelled antibody such as for example, Indium-111 labeled F(ab′)2 fragments of monoclonal antibodies directed against the 17-1A and 19-9 gastrointestinal cancer markers [Watanabe, Y. et al., J. Nuc. Med. (1988), 29: 1436-42] or an immunocytochemistry [e.g., monoclonal antibodies for cytokeratins (Vagunda, V. et al., Eur. J. Cancer, 2001, 37:1847-52) or MIB-1 (Lin, O. et al., Am. J. Clin. Pathol., 2003, 120: 209-16)] which can be visualized via light microscopy and a morphological stain such as DAPI stain which can be visualized via fluorescent microscopy.

(ix) an immunological stain using a radiolabelled antibody such as for example, Indium-111 labeled F(ab′)2 fragments of monoclonal antibodies directed against the 17-1A and 19-9 gastrointestinal cancer markers [Watanabe, Y. et al., J. Nuc. Med. (1988), 29: 1436-42] or an immunocytochemistry [e.g., monoclonal antibodies for cytokeratins (Vagunda, V. et al., Eur. J. Cancer, 2001, 37:1847-52) or MIB-1 (Lin, O. et al., Am. J. Clin. Pathol., 2003, 120: 209-16)] which can be visualized via light microscopy and an activity stain such as cytochemical stain using glutathione-mercury orange complexes (Larrauri, A. et al., J. Histochem. Cytochem. 1987, 35: 271-4) and substrate binding assays stain which can be visualized via fluorescent microscopy.

(x) an immunological stain using a radiolabelled antibody such as for example, Indium-111 labeled F(ab′)2 fragments of monoclonal antibodies directed against the 17-1A and 19-9 gastrointestinal cancer markers [Watanabe, Y. et al., J. Nuc. Med. (1988), 29: 1436-42] or an immunocytochemistry [e.g., monoclonal antibodies for cytokeratins (Vagunda, V. et al., Eur. J. Cancer, 2001, 37:1847-52) or MIB-1 (Lin, O. et al., Am. J. Clin. Pathol., 2003, 120: 209-16)] which can be visualized via light microscopy and fluorescent in situ hybridization (FISH) stain using for example the UroVysion kit probes (Vysis Inc, Downers Grove, Ill., USA) which can be visualized via fluorescent microscopy.

(xi) an immunological stain using a radiolabelled antibody such as for example, Indium-111 labeled F(ab′)2 fragments of monoclonal antibodies directed against the 17-1A and 19-9 gastrointestinal cancer markers [Watanabe, Y. et al., J. Nuc. Med. (1988), 29: 1436-42] or an immunocytochemistry [e.g., monoclonal antibodies for cytokeratins (Vagunda, V. et al., Eur. J. Cancer, 2001, 37:1847-52) or MIB-1 (Lin, O. et al., Am. J. Clin. Pathol., 2003, 120: 209-16)] which can be visualized via light microscopy and a DNA-binding fluorescent dye such as DAPI or Ethidium bromide which can be visualized via fluorescent microscopy.

(xii) an immunological stain using a fluorescently labeled antibody such as fluorescein conjugated anti-p53 (Pantropic) antibody (OP43F, Calbiochem, San Diego, Calif.) which can be visualized via fluorescent microscopy and a morphological stain such as May-Grünwald-Giemsa stain, Giemsa stain, Papanicolau stain, Hematoxylin-Eosin stain which can be visualized via light microscopy.

(xiii) an immunological stain using a fluorescently labeled antibody such as fluorescein conjugated anti-p53 (Pantropic) antibody (OP43F, Calbiochem, San Diego, Calif.) which can be visualized via fluorescent microscopy and an activity stain such as a cytochemical stain (e.g., glucose-6-phosphatase, alkaline phosphatase) and substrate binding assays stain (e.g., using Vector blue) which can be visualized via light microscopy.

(xiv) an immunological stain using a fluorescently labeled antibody such as fluorescein conjugated anti-p53 (Pantropic) antibody (OP43F, Calbiochem, San Diego, Calif.) which can be visualized via fluorescent microscopy and an in situ hybridization stain using radiolabeled probes such ³⁵S-, ³²P-labeled DNA probes, Digoxigenin or biotinylated labeled probes conjugated to either horseradish peroxidase and using substrates such as diaminobenzidine (DAB), tetramethylbenzidine (TMB) or to alkaline phosphatase and using substrates such as APase/fast red which can be visualized via light microscopy.

(xv) an activity stain such as a cytochemical stain (e.g., glucose-6-phosphatase, alkaline phosphatase) and substrate binding assays stain (e.g., using Vector Blue) which can be visualized via light microscopy and a morphological stain such as DAPI stain which can be visualized via fluorescent microscopy.

(xvi) an activity stain such as a cytochemical stain (e.g., glucose-6-phosphatase, alkaline phosphatase) and substrate binding assays stain (e.g., using Vector Blue) which can be visualized via light microscopy and an immunological stain using a fluorescently labeled antibody such as fluorescein conjugated anti-p53 (Pantropic) antibody (OP43F, Calbiochem, San Diego, Calif.) which can be visualized via fluorescent microscopy.

(xvii) an activity stain such as cytochemical stain (e.g., glucose-6-phosphatase, alkaline phosphatase) and substrate binding assays stain (e.g., using Vector Blue) which can be visualized via light microscopy and a fluorescent in situ hybridization (FISH) stain using for example the UroVysion kit probes (Vysis Inc, Downers Grove, Ill., USA) which can be visualized via fluorescent microscopy.

(xviii) an activity stain such as a cytochemical stain (e.g., glucose-6-phosphatase, alkaline phosphatase) and substrate binding assays stain (e.g., using Vector Blue) which can be visualized via light microscopy and a DNA-binding fluorescent dye such as DAPI or Ethidium bromide which can be visualized via fluorescent microscopy.

(xix) an activity stain such as a cytochemical stain using for example glutathione-mercury orange complexes (Larrauri, A. et al., J. Histochem. Cytochem. 1987, 35: 271-4) and substrate binding assay stain which can be visualized via fluorescent microscopy and a morphological stain such as May-Grünwald-Giemsa stain, Giemsa stain, Papanicolau stain, Hematoxylin-Eosin stain which can be visualized via light microscopy.

(xx) an activity stain such as a cytochemical stain using for example glutathione-mercury orange complexes (Larrauri, A. et al., J. Histochem. Cytochem. 1987, 35: 271-4) and a substrate binding assay stain which can be visualized via fluorescent microscopy and an immunological stain using a radiolabelled antibody such as for example, Indium-111 labeled F(ab′)2 fragments of monoclonal antibodies directed against the 17-1A and 19-9 gastrointestinal cancer markers [Watanabe, Y. et al., J. Nuc. Med. (1988), 29: 1436-42] or an immunocytochemistry [e.g., monoclonal antibodies for cytokeratins (Vagunda, V. et al., Eur. J. Cancer, 2001, 37:1847-52) or MIB-1 (Lin, O. et al., Am. J. Clin. Pathol., 2003, 120: 209-16)] which can be visualized via light microscopy.

(xxi) an activity stain such as a cytochemical stain using for example glutathione-mercury orange complexes (Larrauri, A. et al., J. Histochem. Cytochem. 1987, 35: 271-4) and a substrate binding assay stain which can be visualized via fluorescent microscopy and an in situ hybridization stain using radiolabeled probes such as ³⁵S-, ³²P-labeled DNA probes, Digoxigenin or biotinylated labeled probes conjugated to either horseradish peroxidase and using substrates such as diaminobenzidine (DAB), tetramethylbenzidine (TMB) or to alkaline phosphatase and using substrates such as APase/fast red which can be visualized via light microscopy.

(xxii) a cytogenetical stain such as G-banding which can be visualized via light microscopy and an immunological stain using a fluorescently labeled antibody such as fluorescein conjugated anti-p53 (Pantropic) antibody (OP43F, Calbiochem, San Diego, Calif.) which can be visualized via fluorescent microscopy.

(xxiii) a cytogenetical stain such as G-banding which can be visualized via light microscopy and a fluorescent in situ hybridization (FISH) stain using for example the UroVysion kit probes (Vysis Inc, Downers Grove, Ill., USA) which can be visualized via fluorescent microscopy.

(xxiv) a cytogenetical stain such as G-banding which can be visualized via light microscopy and a DNA-binding fluorescent dye such as DAPI or Ethidium bromide which can be visualized via fluorescent microscopy.

(xxv) a cytogenetical stain such as R-banding which can be visualized via fluorescent microscopy and an immunological stain using a radiolabelled antibody such as for example, Indium-111 labeled F(ab′)2 fragments of monoclonal antibodies directed against the 17-1A and 19-9 gastrointestinal cancer markers [Watanabe, Y. et al., J. Nuc. Med. (1988), 29: 1436-42] or an immunocytochemistry [e.g., monoclonal antibodies for cytokeratins (Vagunda, V. et al., Eur. J. Cancer, 2001, 37:1847-52) or MIB-1 (Lin, O. et al., Am. J. Clin. Pathol., 2003, 120: 209-16)] which can be visualized via light microscopy.

(xxvi) a cytogenetical stain such as R-banding which can be visualized via fluorescent microscopy and an in situ hybridization stain using radiolabeled probes such as ³⁵S-, ³²P-labeled DNA probes, Digoxigenin or biotinylated labeled probes conjugated to either horseradish peroxidase and using substrates such as diaminobenzidine (DAB), tetramethylbenzidine (TMB) or to alkaline phosphatase and using substrates such as APase/fast red which can be visualized via light microscopy.

(xxvii) an in situ hybridization stain using radiolabeled probes such as ³⁵S-, ³²P-labeled DNA probes, Digoxigenin or biotinylated labeled probes conjugated to either horseradish peroxidase and using substrates such as diaminobenzidine (DAB), tetramethylbenzidine (TMB) or to alkaline phosphatase and using substrates such as APase/fast red which can be visualized via light microscopy and a DNA-binding fluorescent dye such as DAPI or Ethidium bromide which can be visualized via fluorescent microscopy.

(xxviii) a DNA-binding fluorescent dye such as DAPI or Ethidium bromide which can be visualized via fluorescent microscopy and an in situ hybridization stain using radiolabeled probes such as ³⁵S-, ³²P-labeled DNA probes, Digoxigenin or biotinylated labeled probes conjugated to either horseradish peroxidase and using substrates such as diaminobenzidine (DAB), tetramethylbenzidine (TMB) or to alkaline phosphatase and using substrates such as APase/fast red which can be visualized via light microscopy.

Example 1 of the Examples section which follows provides further description of suitable dual staining dual imaging approaches.

Preferably a single automated device which is capable of processing and integrating a number of different signals is utilized for dual stain visualization. Such a device is preferably capable of simultaneous dual visualization although sequential visualization can also be utilized for sample analysis. An Example of a device suitable for use with the present invention is the Duet™ (Bio View Ltd. Israel).

Thus, the methods of the present invention increase the information which can be obtained from a biological sample and thus improve the accuracy of detection of cancerous cells.

As explained hereinabove, the detection of cancerous cells in biological samples is an important tool for diagnosing cancer. Early detection of cancer can inhibit the progression of cancer to an invasive and less-curable disease, and thus increase the survival rate of the patients at risk.

Thus according to another aspect of the present invention there is provided a method of diagnosing cancer in a subject. As used herein, the phrase “diagnosing cancer in a subject” refers to detecting the presence of cancerous cells in cells derived from the subject, i.e., in biological samples obtained from the subject.

The method is effected by obtaining a biological sample from the subject and processing the sample as described above in order to detect the presence or absence of cancer cells in the sample.

Biological samples can be obtained by any means of sampling a tissue or a body fluid from a subject, such as drawing blood, catheterization of urine, aspiration of fluid, fine needle aspirations, scraping and the like.

The present approach can be utilized for diagnosing numerous types of cancers. In particular, the present approach is highly suitable for detecting transitional cell carcinoma of the bladder since even the low grade tumors include transitional epithelial cells with atypical morphology and abnormal FISH pattern which can be detected in a urine sample using the double staining and dual imaging approach of the present invention.

The term “carcinoma” refers to a malignant epithelial neoplasm which invades the surrounding tissue and metastasizes to distant regions of the body.

Transitional cell carcinoma (TCC) of the bladder is a malignant, usually papillary tumor, derived from transitional stratified epithelium, which occurs most frequently in the bladder. However, most tumors in the collecting system of the human body are transitional cell carcinomas.

Bladder cancer is the fourth most prevalent human malignancy, with about 49,000 new cases and 9,700 deaths reported annually [Silverman, D. T. et al., Epidemiology of Bladder Cancer. In: “Hematology/Oncology Clinics of North America”. P. W. Kantoff et al.. eds., W.B. Saunders Co., Philadelphia, p. 1 (1992)]. Ninety percent of bladder cancers are transitional cell carcinomas which are typically superficial at early stages but often become invasive at later stages, 5% of bladder cancers are squamous cell carcinomas (SCC), which are more prevalent in cases of chronic bladder irritation, and the remainders are rare tumors such as adenocarcinoma, carcinosarcoma.

When applied to TCC detection, the method of the present invention preferably utilizes a urine sample. Such samples are usually obtained via voiding urine or catheterization and contain transitional epithelial cells as well as residual blood cells.

As is illustrated in Examples 1 and 2 of the Examples section which follows, using the teachings of the present invention TCC was diagnosed in several urine samples which were scored as being normal when tested using the cytology detection method alone.

It will be appreciated that positive identification of TCC in a urine sample usually correlates with bladder cancer. Therefore, the method of detecting TCC in a urine sample according to the teachings of the present invention can be accurately utilized for diagnosing individuals having early to late stages of bladder cancer.

Bladder cancer is usually diagnosed via cystoscopy, an invasive procedure, wherein a fiber optic device is inserted into the bladder and lesions are detected visually by a urologist. Cystoscopy is performed on patients expressing the symptom complex characteristic of bladder cancer, i.e., hematuria, pain, or urinary obstruction. However, when symptoms appear, the tumor is usually progressed to a dangerous grade or stage. In addition, this type of macroscopic diagnostics fails to detect microscopic disease such as carcinoma in situ [Halachmi et al., (2001), Bladder cancer: genetic overview. Med. Sci. Monit. 7: 164-168]. Following cystoscopy, a biopsy of the tumor is further examined under a microscope using histological staining methods. However, since such biopsies are limited to small areas of the bladder, some malignant cells can be potentially missed.

Indeed, as is shown in Example 2 of the Example section which follows, bladder biopsies failed to detect TCC in three TCC-positive cases.

Other methods of diagnosing bladder cancer include the analysis of urine samples obtained from patients at risk. In the current practice urine samples are stained with cytological stains as described hereinabove. It will be appreciated that urine samples can be stained using other methods such as immunological stains, DNA image Cytometry (ICM) and FISH stains [Dalquen P. et al., (2002). DNA image cytometry and fluorescence in situ hybridization for noninvasive detection of urothelial tumors in voided urine Cancer. 96: 374-9].

As is further shown in Example 2 of the Examples section which follows, bladder cancer was successfully diagnosed in 26/35 cases using the combined staining/dual imaging method of the present invention. On the other hand, using cytological staining alone, TCC was accurately diagnosed in only 15/35 cases.

It will be appreciated that positive TCC in a urine sample can also suggest the presence of carcinoma in situ, TCC of the kidney and/or TCC of the ureter, all of which can be mis-diagnosed by cystoscopy. Indeed, as is further shown in Table 1 of the Examples section which follows, using the combined staining/dual imaging method TCC was identified in two urine samples of cases with normal cystoscopy findings.

It is expected that during the life of this patent many relevant staining methods will be developed and the scope of the term staining is intended to include all such new technologies a priori.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

Transitional Cell Carcinoma is Accurately Detected Using Double Staining and Dual Imaging

In order to test the suitability of the combined staining/dual imaging method of the present invention in identifying transitional cell carcinoma (TCC), voided urine samples were subjected to morphology staining followed by FISH analysis and the staining results were analyzed using dual imaging.

Materials and Experimental Methods

Preparation of cytospin slides of voided urine samples—Voided urine samples (volume ranged from 4 ml to 45 ml, mean 15±11.3 ml) were centrifuged at room temperature for 10 minutes at 300×g. Following centrifugation cell pellets were resuspended in 100-300 μl of a Morphology Preserver (BioWhite kit, BioView LtD., Rehovot, Israel) and the concentration of cells was determined using a Neubauer improved counting chamber (Neubauer, Germany). Cells were then cytospun at a cell density of 300-500 cells per mm² according to manufacturer's instructions (Kubota, Japan). Cytospin slides were fixed for 48 hours in 95% ethanol at room temperature, wrapped with aluminum foil and kept at −20° C.

Morphology staining—For morphological observations, cytospin slides were stained with May-Grünwald-Giemsa which labels the nucleus in deep purple and the cytoplasm in various shades from pink to light purple. Slides were dipped in May-Grünwald stain (Cat. # MAY-1, Sigma-Aldrich Corp., St Louis, Mo., USA) for 3 minutes, rinsed in distilled water and dipped in a diluted (1:20 in distilled water) Giemsa stain (Cat. # GS-500, Sigma-Aldrich Corp.) for 7 minutes. Slides were then rinsed under running tap water and air-dried.

FISH probes—Two different mixes of FISH probes were used: Mix I, which includes DNA probes of the pericentromeric regions of chromosome 3 (labeled in red), chromosome 7 (labeled in green) and chromosome 17 (labeled in aqua) available from Qbiogene, Illkirch Cedex, France, and Mix II, the UroVysion kit, which includes DNA probes of the pericentromeric regions of chromosome 3 (labeled in red), chromosome 7 (labeled in green), chromosome 17 (labeled in aqua), and to the 9p21 locus of chromosome 9 (labeled in gold) available from Vysis Inc, Downers Grove, Ill., USA.

Fluorescent In Situ Hybridization (FISH)—FISH analysis was performed on slides previously stained with May-Grünwald-Giemsa. Slides were de-stained and fixed for one hour in an ice-cold methanol: acetic acid (3:1) solution, rinsed twice, 5 minutes each, in phosphate buffered saline (PBS) at room temperature and air-dried. Slides were then digested for 15 minutes in a warm solution (at 37° C.) of 0.05% digestion enzyme (BioBlue kit, BioView Ltd., Rehovot, Israel) in 10 mM HCl. Following digestion, slides were rinsed for 5 minutes in PBS, fixed in an ice-cold methanol: acetic acid (3:1) solution, washed for 5 minutes in PBS and dehydrated for 2 minutes in a series of ice-cold 70, 80 and 100% ethanol solutions. Prior to hybridization, slides and FISH probes were co-denatured at 74° C. for 4 minutes in a solution of 70% formamide. Hybridization was performed for overnight at 37° C. in a moist chamber according to probe's manufacturer's instructions. Following hybridization slides were rinsed for 2 minutes in a sodium chloride/sodium citrate solution (60 mM /6 mM), respectively. For a complete removal of excess of probes, slides were further washed for 2 minutes at room temperature in a sodium chloride/sodium citrate/NP-40 solution (300 mM /30 mM /0.1%), respectively. After tapping off the excess wash solution, 10 μl of the Blue View counterstain (Bio View Cat. # BV-002-002) was employed and slides were covered with 22×50 mm coverslips and maintained at −20° C. in the dark.

Microscopic analysis using the Bio View Duet™ system—Following a morphological staining, slides were automatically scanned using the ×20 dry objective of the BioView Duet™ system (Bio View Ltd, Rehovot, Israel). This system is based on a dual mode, fully automated microscope (Axioplan 2, Carl Zeiss, Jena, Germany), an XY motorized 8-slides stage (Marzhauser, Wetzler, Germany), a 3CCD (charged coupled device) progressive scan color camera (DXC9000, Sony, Tokyo, Japan), and a computer for control and analysis of the data. The system has a unique feature of allowing the scanning of the same slide twice using both a morphological stain and a FISH stain. The coordinates and images of all cells found in the first scan, including the abnormal and/or suspicious cells were saved prior to the second scan. The second scan was performed following the FISH stain using the ×63 dry objective and the appropriate filters, while producing a combined image of both stains. Combined images were then automatically classified into predefined classes as described hereinunder.

Scoring methodology of samples obtained using the combined staining method—Following the morphology staining a minimum of 25 and a maximum of 260 cells were selected per slide. If less than 25 morphology atypical cells were found, a random FISH scan of at least 100 cells was performed. Samples were defined as technically unsuccessful if fewer than 25 cells were found and analyzed by the system. Samples were diagnosed as TCC-positive if included at least one cell with both abnormal morphology and abnormal FISH pattern. Cells exhibiting gains of at least two chromosomes were scored as abnormal. When abnormal FISH pattern was observed in morphological normal cells then a minimum of five FISH-abnormal cells were required for positive TCC diagnosis. In slides hybridized to probe mix II, the previous mentioned criteria or the loss of the 9p21 locus in at least 12 cells was required for a positive diagnosis regardless of cell morphology.

Scoring methodology of the cytology, cystoscopy and biopsy results—Cytology slides were scored according to the following categories: Class I—normal, Class II—inflammation, Class III—suspicious for malignancy, and Class IV—malignant. Cystoscopy findings were scored as papillary lesions highly suspicious for TCC (“positive”, table 1, hereinbelow), lesions of uncertain significance (“suspicious” table 1, hereinbelow), or negative. Bladder biopsies or transurethral resection of bladder tumor (TURBT) scored as positive, suspicious or negative for bladder cancer and their grade and stage were determined according to pathological tumor/node/metastasis (TNM) pT criteria (American Joint Committee on Cancer: AJCC Cancer Staging Manual, 5^{th} ed. Edited by I. D. Fleming. Philadelphia: Lippincott-Raven, pp. 303-314, 1997).

Experimental Results

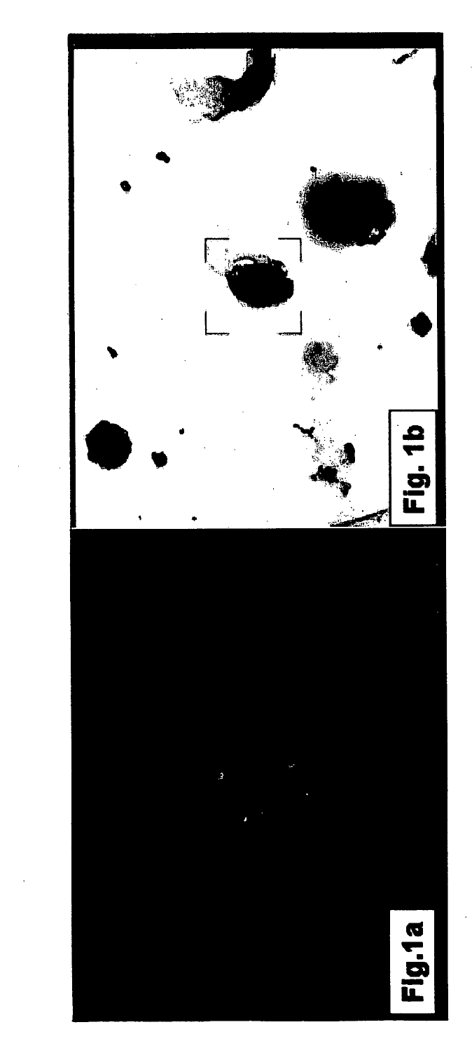

The identification of transitional cell carcinoma based on abnormal morphology and chromosome 3, 17 and 18 polyploidy—Urine sample cells were subjected to May-Grünwald-Giemsa stain and the morphology of the transitional epithelial cells was evaluated under light microscopy using an automated cell scanning system (BioView Duet™, Bio View, Ltd, Rehovot, Israel). As is shown in FIGS. 1a-b, following May-Grünwald-Giemsa stain a suspicious epithelial cell was identified exhibiting a high nucleus to cytoplasm ratio and an irregular nucleus (FIG. 1b, cell marked with square brackets). When the sample was further subjected to FISH analysis and DAPI counterstaining the same cell exhibited a patchy nucleus using the DAPI stain (FIG. 1a, blue counterstain) with gains of chromosomes 3, 7 and 17 (FIG. 1a, red, green and aqua signals, respectively). Thus, the FISH results confirmed the findings of the cytological staining. These results suggest the use of dual imaging for a confirmative diagnosis of bladder cancer in voided urine samples.

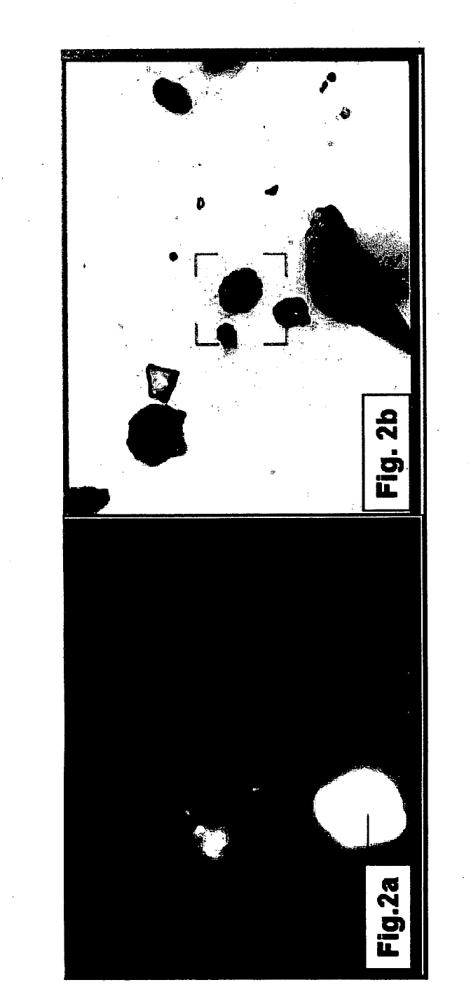

Transitional cell carcinoma can be ruled out based on triple staining and dual imaging—Transitional epithelial cells were screened for the presence of TCC in voided urine samples. Generally, in addition to urine cytology and FISH analysis patients are offered to go through a cystoscopy in regular intervals in order to rule out the presence of malignant bladder epithelial cells. In the present study, voided urine samples were subjected to both cytology evaluation using May-Grünwald-Giemsa stain and FISH analysis and were evaluated using the BioView Duet™ system. FIGS. 2a-b demonstrate an example of a urine sample with a morphological suspicious cell using DAPI stain (FIG. 2b, blue counterstain), however with normal morphology using May-Grünwald-Giemsa stain (FIG. 2b, cell marked with square brackets) and normal karyotype using FISH stain (FIG. 2a, red, green and aqua signals). In this case TCC was ruled out without subjecting the patient to unnecessary cystoscopy.

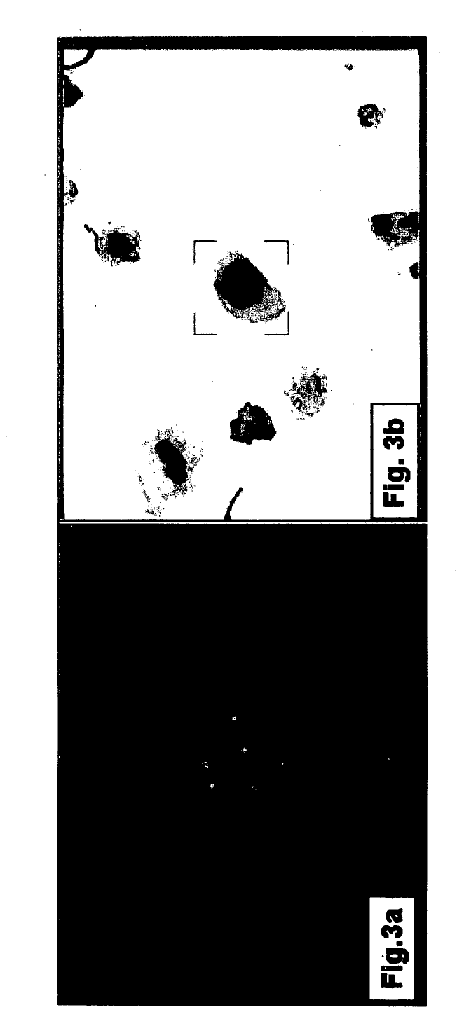

The identification of TCC in a morphologically normal transitional epithelial cell—Transitional epithelial cells were screened for TCC using both DAPI and May-Grünwald-Giemsa stains. As is shown in FIG. 3b, the epithelial cells in the sample exhibited a slightly atypical morphology which was yet inconclusive regarding the presence of TCC. However, subsequent FISH analysis revealed an abnormal karyotype with multiple gains of chromosomes 3, 7 and 17 and (FIG. 3a, red, green and aqua signals, respectively). Thus, using the combined staining method and dual imaging TCC was identified in the voided urine sample.

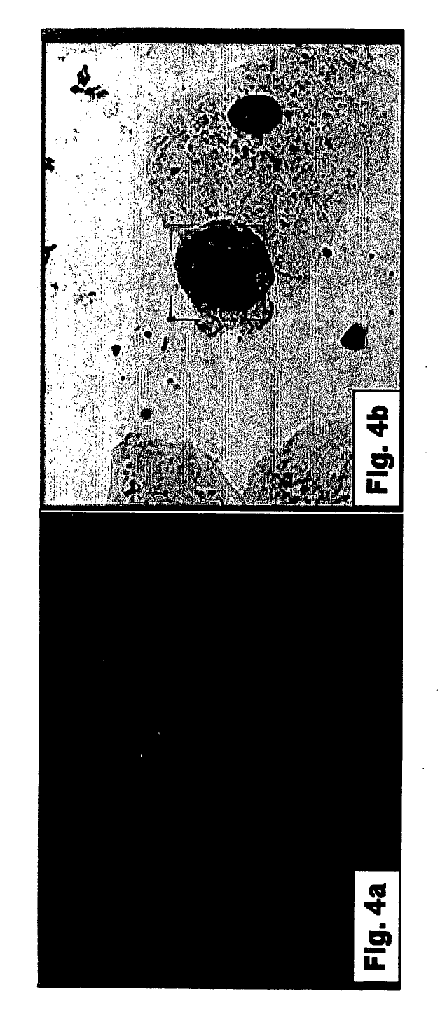

The detection of rare, abnormal epithelial cells based on dual stains and dual imaging—Transitional epithelial cells were screened for TCC using May-Grünwald-Giemsa and FISH stains. As is shown in FIGS. 4a-b, the combined staining method and dual imaging of the present invention enabled the identification of a single abnormal TCC cell in a voided urine sample. This cell exhibited an abnormal morphology with a high nucleus to cytoplasm ratio and a dark May-Grünwald-Giemsa stain (FIG. 4b 6l ) as well as gains of chromosomes 3, 7 and 17 (FIG. 4a, red, green and aqua signals, respectively) as detected using FISH analysis. Noteworthy is that in cases like this, based on the current practice set of standards, the presence of only one cell with a suspicious morphology is not indicative of TCC. Moreover, the chances of locating such a cell while scanning the slide manually are very low. Thus, these findings demonstrate that using the combined staining/dual imaging method of the present invention it is possible to identify rare cancer cells which are practically undetectable using prior art methods. .

### Example 2

A Combined Dual Staining/Dual Imaging Method is Highly Sensitive in Diagnosing TCC

In order to test the sensitivity of the combined staining/dual imaging method of the present invention in diagnosing transitional cell carcinoma (TCC), the diagnostic scores obtained using this method were compared with those obtained by urine cytology, cystoscopy and bladder biopsies.

Experimental and Statistical Results

Comparative analysis of screening methods for transitional cell carcinoma (TCC)—Thirty five urine samples were screened for the presence of TCC using either a morphological stain alone (see “cytology” in Table 1, hereinbelow) or a combined morphological/FISH dual imaging method (see “combined” in Table 1, hereinbelow). The accuracy of TCC diagnosis was compared to concurrent cystoscopy findings and pathological examination of bladder biopsies.

TABLE 1
Comparison of various detection methods for transitional cell carcinoma

| | Mix I | | Mix II | | | | | | | |
|---|---|---|---|---|---|---|---|---|---|---|
| | No. of | | No. of | | | | | | | |
| | abnorm. | | abnorm. | | | | | | | |
| | cells by | | cells by | | | | | | | |
| | FISH | | FISH | | | Pathology | | | | |
| I.D. | (Morph.) | Comb. | (Morph.) | Comb. | Cytol. | Diagnos. | Grade | Stage | Cystoscopy | History |
| B-158 | 22 (22) | Pos. | — | — | Pos. | Pos. | 3 | pT2 | Sus. | Y |
| B-160 | 76 (76) | Pos. | 77 (77) | Pos. | Pos. | Pos. | 2 | pT1 | Pos. | N |
| B-170 | 80 (80) | Pos. | 43 (43) | Pos. | Pos. | Pos. | 3 | pT3 | Pos. | Y |
| B-171 | 8 (8) | Pos. | — | — | Pos. | Pos. | 2 | pT2 | Pos. | Y |
| B-175 | 26 (24) | Pos. | 4 (4) | Pos. | Pos. | Pos. | 3 | pT2 | Pos. | Y |
| B-176 | 6 (5) | Pos. | — | — | Pos. | Pos. | 1 | pTa | Pos. | N |
| B-179 | 69 (69) | Pos. | — | — | Pos. | Pos. | 3 | pT2 | Sus. | N |
| B-194 | 28 (28) | Pos. | — | — | Pos. | Pos. | 2 | pT1 | Pos. | Y |
| B-198 | 113 (113) | Pos. | — | — | Pos. | Pos. | 2-3 | pT3 | Pos. | N |
| B-199 | 240 (240) | Pos. | — | — | Pos. | Pos. | 3 | pT3 | Pos. | Y |
| B-202 | 17 (17) | Pos. | — | — | Pos. | Pos. | 1 | pTa | Sus. | Y |
| B-208 | — | — | 122 (122) | Pos. | Pos. | Pos. | 2 | pT1 | Pos. | N |
| B-157 | 23 (23) | Pos. | 35 (34) | Pos. | Pos. | Neg. | — | — | Sus. | Y |
| B-159 | 3 (3) | Pos. | — | — | Pos. | Pos. | 1 | pTa | Pos. | Y |
| B-174 | — | — | 2 (2) | Pos. | Pos. | Neg. | — | — | Pos. | Y |
| B-164 | 5 (4) | Pos. | 2 (2) | Pos. | Neg. | Pos. | 1 | pTa | Sus. | N |
| B-169 | 21 (21) | Pos. | 14 (14) | Pos. | Neg. | Neg. | — | — | Neg. | Y |
| B-178 | 7 (6) | Pos. | 8 (8) | Pos. | Neg. | Neg. | — | — | Neg. | Y |
| B-180 | 5 (3) | Pos. | — | — | Neg. | Pos. | 1 | pTa | Pos. | Y |
| B-197 | 34 (33) | Pos. | 39 (39) | Pos. | Neg. | — | — | — | Pos. | N |
| B-149 | — | — | 2 (2) | Pos. | — | Pos. | 1-2 | pTa | Pos. | Y |
| B-156 | 1 (1) | Pos. | 1 (1) | Pos. | Neg. | Pos. | 1 | pTa | Sus. | Y |
| B-163 | 4 (4) | Pos. | — | — | Neg. | Pos. | 1 | pTa | Pos. | Y |
| B-165 | 2 (1) | Pos. | — | — | Neg. | Pos. | 1 | pTa | Pos. | Y |
| B-172 | 2 (2) | Pos. | 4 (4) | Pos. | — | Pos. | 1 | pTa | Pos. | Y |
| B-192 | 0 | Neg. | 84 (0) | Pos. | — | Pos. | 1 | pTa | Sus. | Y |
| B-191 | 3 (0) | Neg. | — | — | Neg. | Neg. | — | — | Neg. | Y |
| B-154 | 0 | Neg. | — | — | Neg. | Neg. | — | — | Sus. | N |
| B-155 | 1 (0) | Neg. | — | — | Neg. | — | — | — | Pos. | Y |
| B-166 | 0 | Neg. | 2 (0) | Neg. | Neg. | Neg. | — | — | Neg. | Y |
| B-181 | 1 (0) | Neg. | — | — | Neg. | Neg. | — | — | Sus. | N |
| B-182 | 2 (0) | Neg. | — | — | Neg. | Neg. | — | — | Sus. | N |
| B-145 | 1 (0) | Neg. | — | — | — | Neg. | — | — | Sus. | Y |
| B-148 | — | — | 0 | Neg. | Neg. | Neg. | — | — | Neg. | N |
| B-210 | — | — | 3 (0) | Neg. | Neg. | — | — | — | Neg. | N |

A comparison of TCC diagnosis using a morphological stain (cytology), a combined FISH/morphological dual imaging method (combined), a pathological evaluation of bladder biopsies (pathology) and a bladder cystoscopy.
I.D. = case identification number,
Mix I = FISH probes from the pericentromeric regions of chromosomes 3, 7 and 17,
Mix II = FISH probes from the pericentromeric regions of chromosomes 3, 7, 17 and 9p21,
Comb. = combined,
abnorm. = abnormal,
Morph. = morphology,
Cytol. = cytology,
Diagnos. = diagnosis,
Pos. = positive,
Neg. = negative,
Sus. = suspicious,
Y = yes,
N = no.

As is shown in Table 1 hereinabove, using the combined staining/dual imaging method of the present invention, i.e., morphological staining followed by FISH, TCC was diagnosed in 26 urine samples and was ruled out in 9 samples. Pathological evaluations of bladder biopsies confirmed the diagnosis of TCC in 21 out of the 26 cases which were scored as “positive” using the combined staining method (Table 1, hereinabove). Noteworthy is that four biopsy-negative cases (B-157, B-169, B-174, B-178) had a history of biopsy-proved bladder cancer, and in one of them (B-169), the recurrence of the disease was noticed during a subsequent cystoscopy which was performed six months later. In three biopsy-negative samples (B-157, B-169 and B-178), the diagnosis of TCC using the combined staining method was based on the presence of at least seven TCC-suspected cells (i.e., cells with both abnormal FISH signals and abnormal morphology) in each sample, a finding which correlates with the presence of TCC. These results suggest that TCC can be diagnosed in urine samples prior to its diagnosis in bladder biopsies. In another biopsy-negative case (B-174), although abnormal FISH signals were found in only two cells, the morphological staining revealed the presence of multiple abnormal cells, a finding which demonstrates the power of the combined staining method over the FISH staining method alone in detecting TCC.

As is further shown in Table 1 hereinabove, in 7 out of the 9 cases which were scored as “negative” using the combined staining method, subsequent pathological evaluation of bladder biopsies have confirmed the absence of TCC. Noteworthy is that biopsy was not performed in the other two TCC-negative cases. Thus, these results demonstrate that the combined staining method is 100% accurate in ruling out TCC using urine samples.

When urine samples were scored according to cell morphology alone (see “Cytology”, Table 1 hereinabove), TCC was diagnosed in only 15 out of the 26 samples which were scored as “positive” using the combine staining method. The other 11 urine samples included cells exhibiting either a normal morphology or a morphology typical of inflammation, and as such were scored as “negative” for the presence of TCC. In four samples (B-145, B-149, B-172, B-192) the cytology analysis failed due to insufficient abnormal cells in the sample. However, as is further shown in Table 1 hereinabove, in one of these samples, sample B-192, the combined staining method detected the presence of 84 cells with abnormal FISH signals, a finding which correlates with the presence of TCC. Indeed, a subsequent biopsy confirmed the diagnosis of TCC in B-192. Thus, these results demonstrate that the combined staining method of the present invention is superior to the cytology method in diagnosing bladder cancer.

When the combined staining method was compared with the results obtained using cystoscopy it was found that of the 26 “TCC-positive” cases according to the combined method, cystoscopy detected papillary lesions highly suspicious for TCC in 17 cases, lesions of uncertain significance in 7 cases and normal findings in two cases (Table 1, hereinabove). On the other hand, in cases with negative diagnosis based on the combined staining method, cystoscopy revealed one case with lesions highly suspicious for TCC (B-155), 4 cases with lesions of uncertain significance (B-154, B-181, B-182, B-145) and 4 cases with normal findings (Table 1, hereinabove). It is noteworthy, that in cases B-155, B-154, B-181, B-182 and B-145, the negative diagnosis of TCC using the combined method was based on the presence of a maximum of two cells with abnormal FISH signals, yet with normal morphology, which is insufficient for TCC diagnosis in urine samples.

Altogether, these results suggest the suitability of combined staining/dual imaging method of the present invention in early diagnosis of TCC in urine samples.

The combined staining method can accurately detect TCC in biopsy-positive cases—The sensitivity of the combined staining/dual imaging method of the present invention was further compared with that of the cytology method in biopsy-positive TCC cases. As is shown in Table 2 hereinbelow, while the combined staining method detected TCC in urine samples of all cases with stage pTa tumors, the cytology method detected TCC in only 3 out of the 11 cases (p<0.05). On the other hand, a similar detection level was found in TCC cases with stage pT1-4 tumors using both the combined method and the cytology method. In addition, while the combined staining method was capable of detecting TCC in urine samples of all cases with grade 1 and 2 tumors, the cytology method detected TCC in only 30% of cases with grade 1 tumors and in 80% of cases with grade 2 tumors. Noteworthy is that both the cytology method and the combined staining/dual imaging method detected TCC in all cases with grade 3 tumors. Thus, as is further shown in Table 2 hereinbelow, while the overall sensitivity of the cytology method in detecting TCC in urine samples of biopsy-positive cases was approximately 60%, the overall sensitivity of the combined method was 100% (p<0.05).

TABLE 2
Sensitivity and specificity of TCC detection in
urine samples in biopsy-positive TCC cases

No. of detected cases/total
cases (%)

| | Combined | | |
|---|---|---|---|
| | analysis | Cytology | p Values |
| Stage | | | |
| pTa | 11/11 (100%) | 3/11 (27.3%) | 0.0133 |
| pT1-4 | 10/10 (100%) | 10/10 (100%) | — |
| Grade | | | |
| 1 | 10/10 (100%) | 3/10 (30%) | 0.023 |
| 2 | 5/5 (100%) | 4/5 (80%) | 1 |
| 3 | 6/6 (100%) | 6/6 (100%) | — |
| Overall | 21/21 (100%) | 13/21 (61.9%) | 0.0133 |
| sensitivity | | | |

Frequencies of TCC cases detected in urine samples using the cytology method alone, or the combined staining/dual imaging method in biopsy-positive TCC cases. P values reflect the significance of differences between the combined staining method and the cytology method.

The combined staining method of the present invention is more specific than prior art methods in detecting TCC in urine samples of biopsy-positive cases—The sensitivity of the combined staining/dual imaging method of the present invention in detecting TCC in biopsy-positive cases was compared with the sensitivity observed using prior art methods. These included the methods described by Halling et al., 2000, J Urol, 164: 1768, and Bubendorf at al., 2001, Am J Clin Pathol, 116: 79, which are based on scanning for cells with nuclear abnormalities under DAPI staining and determining the FISH pattern in those cells, or the method described by Skacel et al., 2003, J Urol, 169: 2101, in which FISH was performed on cytology archival slides while selecting and marking cytologically atypical cells.

As is shown in Table 3 hereinbelow, while 100% of TCC cases with stage pTa tumors were accurately diagnosed using the combined method of the present invention only 65-83% of the cases with the same stage tumor were diagnosed using the prior art methods. Similarly, the combined method was far more sensitive in detecting TCC in cases with grade 1 and 2 tumors. Thus, while 100% of cases with grade 1 and 2 tumors were diagnosed using the combined method of the present invention, only 36-86% of the cases with similar grade tumors were diagnosed using the prior art methods (Table 3, hereinbelow). Thus, as is further shown in Table 3 hereinbelow, the overall sensitivity and specificity of the combined method of the present invention is far higher than those of prior art methods.

TABLE 3
Sensitivity and specificity of TCC detection in urine
samples using the combined staining/dual imaging
method of the present invention as compared with
prior art approaches

| | No. of detected cases/total cases (%) | | | |
|---|---|---|---|---|
| | Combined | Halling et al. | Bubendorf et | Skacel et al. |
| | analysis | (2000) | al. (2001) | (2003) |
| Stage | | | | |
| PTa | 11/11 (100) | 24/37 (65) | 33/45 (73) | 53/64 (83) |
| pT1-4 | 9/9 (100) | 18/19 (95) | 15/15 (100) | 14/15 (93.3) |
| Grade | | | | |
| 1 | 10/10 (100) | 4/11 (36) | 15/21 (71) | 19/23 (83) |
| 2 | 5/5 (100) | 19/25 (76) | 25/29 (86) | 28/35 (80) |
| 3 | 6/6 (100) | 36/37 (97) | 16/17 (94) | 23/24 (96) |
| Overall | 21/21 (100) | 59/73 (81) | 56/67 (83.6) | 70/82 (85) |
| sensitivity | | | | |
| Specificity | 6/6 (100) | 75/78 (96) | 58/60 (96) | 28/29 (97) |

Frequencies of TCC detection in urine samples in biopsy-positive TCC cases as determined using the combined staining method of the present invention, or the methods disclosed in Halling et al., (2000), J Urol, 164: 1768, Bubendorf et al., (2001), Am J Clin Pathol, 116: 79, Skacel et al. (2003), J Urol, 169: 2101. Specificity was calculated for patients with no history of bladder cancer and a negative cystoscopy.

Therefore, these results demonstrate that the combined staining/dual imaging method of the present invention is more accurate, sensitive and specific than prior art approaches and thus is better suited for detection of TCC and bladder cancer as well as other cancers.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of identifying cancerous cells in a biological sample comprising:
(a) staining nucleated cells of the biological sample with at least two stains to thereby obtain stained nucleated cells, and;
(b) sequentially and/or simultaneously exposing said stained nucleated cells to at least two imaging modes, to thereby identify the cancerous cells in the biological sample.

2. The method of claim 1, wherein each imaging mode of said at least two imaging modes is specific to a stain of said at least two stains.

3. The method of claim 1, wherein the cancerous cells are associated with a cancer selected from the group consisting of leukemia, lymphoma, brain cancer, cerebrospinal cancer, bladder cancer, prostate cancer, breast cancer, cervix cancer, uterus cancer, ovarian cancer, kidney cancer, esophagus cancer, lung cancer, colon cancer, pancreatic cancer, and melanoma.

4. The method of claim 1, wherein the biological sample is selected from the group consisting of bone marrow cells, lymph nodes cells, peripheral blood, cerebrospinal fluid, urine, effusions, fine needle aspirates, peripheral blood scrapings, paraffin embedded tissues, and frozen sections.

5. The method of claim 1, wherein each stain of said at least two stains is independently selected from the group consisting of a morphological stain, an immunological stain, an activity stain, a cytogenetical stain, in situ hybridization stain and a DNA stain.

6. The method of claim 5, wherein said morphological stain is selected from the group consisting of May-Grünwald-Giemsa stain, Giemsa stain, Papanicolau stain, Hematoxylin-Eosin stain and DAPI stain.

7. The method of claim 5, wherein said immunological stain is selected from the group consisting of fluorescently labeled immunohistochemistry, radiolabeled immunohistochemistry and immunocytochemistry.

8. The method of claim 5, wherein said activity stain is selected from the group consisting of cytochemical stain and substrate binding assay stain.

9. The method of claim 5, wherein said cytogenetical stain is selected from the group consisting of G-banding stain, R-banding stain, Q-banding stain, and C-banding stain.

10. The method of claim 5, wherein said in situ hybridization stain is selected from the group consisting of fluorescent in situ hybridization (FISH) stain, radiolabeled in situ hybridization stain, Digoxigenin labeled in situ hybridization stain and biotinylated in situ hybridization stain.

11. The method of claim 5, wherein said DNA stain is a DNA-binding fluorescent dye.

12. The method of claim 1, wherein a first stain of said at least two stains is a morphological stain and a second stain of said at least two stains is selected from the group consisting of an immunological stain, an activity stain, an in situ hybridization stain, and a DNA stain.

13. The method of claim 1, wherein a first stain of said at least two stains is an immunological stain and a second stain of said at least two stains is selected from the group consisting of a morphological stain, an activity stain, an in situ hybridization stain, and a DNA stain.

14. The method of claim 1, wherein a first stain of said at least two stains is an activity stain and a second stain of said at least two stains is selected from the group consisting of a morphological stain, an immunological stain, an in situ hybridization stain, and a DNA stain.

15. The method of claim 1, wherein a first stain of said at least two stains is a cytogenetical stain and a second stain of said at least two stains is selected from the group consisting of an immunological stain, an in situ hybridization stain, and a DNA stain.

16. The method of claim 1, wherein a first stain of said at least two stains is an in situ hybridization stain and a second stain of said at least two stains is a DNA stain.

17. The method of claim 1, wherein a first stain of said at least two stains is a DNA stain and a second stain of said at least two stains is an in situ hybridization stain.

18. The method of claim 1, wherein step (b) is effected using an automated cell imaging device capable of at least dual imaging.

19. A method of diagnosing cancer in a subject, the method comprising:
(a) obtaining a biological sample from the subject;
(b) staining nucleated cells of said biological sample with at least two stains to thereby obtain stained nucleated cells, and;
(c) sequentially and/or simultaneously exposing said stained nucleated cells to at least two imaging modes, to thereby determine the presence or absence of cancerous cells within said stained nucleated cells, wherein presence of said cancerous cells is indicative of a positive cancer diagnosis.

20. The method of claim 19, wherein each imaging mode of said at least two imaging modes is specific to a stain of said at least two stains.

21. The method of claim 19, wherein the cancer is selected from the group consisting of leukemia, lymphoma, brain cancer, cerebrospinal cancer, bladder cancer, prostate cancer, breast cancer, cervix cancer, uterus cancer, ovarian cancer, kidney cancer, esophagus cancer, lung cancer, colon cancer, pancreatic cancer, and melanoma.

22. The method of claim 19, wherein said biological sample is selected from the group consisting of bone marrow cells, lymph nodes cells, peripheral blood, cerebrospinal fluid, urine, effusions, fine needle aspirates and/or peripheral blood scrapings, paraffin embedded tissues, and frozen sections.

23. The method of claim 19, wherein each stain of said at least two stains is independently selected from the group consisting of a morphological stain, an immunological stain, an activity stain, a cytogenetical stain, in situ hybridization stain and a DNA stain.

24. The method of claim 23, wherein said morphological stain is selected from the group consisting of May-Grünwald-Giemsa stain, Giemsa stain, Papanicolau stain, Hematoxylin-Eosin stain and DAPI stain.

25. The method of claim 23, wherein said immunological stain is selected from the group consisting of fluorescently labeled immunohistochemistry, radiolabeled immunohistochemistry and immunocytochemistry.

26. The method of claim 23, wherein said activity stain is selected from the group consisting of cytochemical stain and substrate binding assay stain.

27. The method of claim 23, wherein said cytogenetical stain is selected from the group consisting of G-banding stain, R-banding stain, Q-banding stain, and C-banding stain.

28. The method of claim 23, wherein said in situ hybridization stain is selected from the group consisting of fluorescent in situ hybridization (FISH) stain, radiolabeled in situ hybridization stain, Digoxigenin labeled in situ hybridization stain and biotinylated in situ hybridization stain.

29. The method of claim 23, wherein said DNA stain is a DNA-binding fluorescent dye.

30. The method of claim 19, wherein a first stain of said at least two stains is a morphological stain and a second stain of said at least two stains is selected from the group consisting of an immunological stain, an activity stain, an in situ hybridization stain, and a DNA stain.

31. The method of claim 19, wherein a first stain of said at least two stains is an immunological stain and a second stain of said at least two stains is selected from the group consisting of a morphological stain, an activity stain, an in situ hybridization stain, and a DNA stain.

32. The method of claim 19, wherein a first stain of said at least two stains is an activity stain and a second stain of said at least two stains is selected from the group consisting of a morphological stain, an immunological stain, an in situ hybridization stain, and a DNA stain.

33. The method of claim 19, wherein a first stain of said at least two stains is a cytogenetical stain and a second stain of said at least two stains is selected from the group consisting of an immunological stain, an in situ hybridization stain, and a DNA stain.

34. The method of claim 19, wherein a first stain of said at least two stains is an in situ hybridization stain and a second stain of said at least two stains is a DNA stain.

35. The method of claim 19, wherein a first stain of said at least two stains is a DNA stain and a second stain of said at least two stains is an in situ hybridization stain.

36. The method of claim 19, wherein step (b) is effected using an automated cell imaging device capable of at least dual imaging.

37. A method of identifying transitional cell carcinoma cells in a urine sample comprising:
(a) staining nucleated cells of the urine sample with at least two stains to thereby obtain stained nucleated cells, and;
(b) sequentially and/or simultaneously exposing said stained nucleated cells to at least two imaging modes, to thereby identify the transitional cell carcinoma cells in the urine sample.

38. The method of claim 37, wherein each imaging mode of said at least two imaging modes is specific to a stain of said at least two stains.

39. The method of claim 37, wherein the transitional cell carcinoma cells are associated with bladder cancer and/or kidney cancer.

40. The method of claim 37, wherein the urine sample is obtained via voided urine or catheterization.

41. The method of claim 37, wherein each stain of said at least two stains is independently selected from the group consisting of a morphological stain, an immunological stain, an activity stain, a cytogenetical stain, in situ hybridization stain and a DNA stain.

42. The method of claim 41, wherein said morphological stain is selected from the group consisting of May-Grünwald-Giemsa stain, Giemsa stain, Papanicolau stain, Hematoxylin-Eosin stain and DAPI stain.

43. The method of claim 41, wherein said immunological stain is selected from the group consisting of fluorescently labeled immunohistochemistry, radiolabeled immunohistochemistry and immunocytochemistry.

44. The method of claim 41, wherein said activity stain is selected from the group consisting of cytochemical stain and substrate binding assay stain.

45. The method of claim 41, wherein said cytogenetical stain is selected from the group consisting of G-banding stain, R-banding stain, Q-banding stain, and C-banding stain.

46. The method of claim 41, wherein said in situ hybridization stain is selected from the group consisting of fluorescent in situ hybridization (FISH) stain, radiolabeled in situ hybridization stain, Digoxigenin labeled in situ hybridization stain and biotinylated in situ hybridization stain.

47. The method of claim 41, wherein said DNA stain is a DNA-binding fluorescent dye.

48. The method of claim 37, wherein a first stain of said at least two stains is a morphological stain and a second stain of said at least two stains is selected from the group consisting of an immunological stain, an activity stain, an in situ hybridization stain, and a DNA stain.

49. The method of claim 37, wherein a first stain of said at least two stains is an immunological stain and a second stain of said at least two stains is selected from the group consisting of a morphological stain, an activity stain, an in situ hybridization stain, and a DNA stain.

50. The method of claim 37, wherein a first stain of said at least two stains is an activity stain and a second stain of said at least two stains is selected from the group consisting of a morphological stain, an immunological stain, an in situ hybridization stain, and a DNA stain.

51. The method of claim 37, wherein a first stain of said at least two stains is a cytogenetical stain and a second stain of said at least two stains is selected from the group consisting of an immunological stain, an in situ hybridization stain, and a DNA stain.

52. The method of claim 37, wherein a first stain of said at least two stains is an in situ hybridization stain and a second stain of said at least two stains is a DNA stain.

53. The method of claim 37, wherein a first stain of said at least two stains is a DNA stain and a second stain of said at least two stains is an in situ hybridization stain.

54. The method of claim 37, wherein step (b) is effected using an automated cell imaging device capable of at least dual imaging.

55. A method of diagnosing bladder cancer in a subject, the method comprising:
(a) obtaining a urine sample from the subject;
(b) staining nucleated cells of said urine sample with at least two stains to thereby obtain stained nucleated cells, and;
(c) sequentially and/or simultaneously exposing said stained nucleated cells to at least two imaging modes, to thereby determine the presence or absence of cancerous cells within said stained nucleated cells, wherein presence of said cancerous cells is indicative of a positive cancer diagnosis.

56. The method of claim 55, wherein each imaging mode of said at least two imaging modes is specific to a stain of said at least two stains.

57. The method of claim 55, wherein the urine sample is obtained via voided urine or catheterization.

58. The method of claim 55, wherein each stain of said at least two stains is independently selected from the group consisting of a morphological stain, an immunological stain, an activity stain, a cytogenetical stain, in situ hybridization stain and a DNA stain.

59. The method of claim 58, wherein said morphological stain is selected from the group consisting of May-Grünwald-Giemsa stain, Giemsa stain, Papanicolau stain, Hematoxylin-Eosin stain and/or DAPI stain.

60. The method of claim 58, wherein said immunological stain is selected from the group consisting of fluorescently labeled immunohistochemistry, radiolabeled immunohistochemistry and immunocytochemistry.

61. The method of claim 58, wherein said activity stain is selected from the group consisting of cytochemical stain and substrate binding assay stain.

62. The method of claim 58, wherein said cytogenetical stain is selected from the group consisting of G-banding stain, R-banding stain, Q-banding stain, and C-banding stain.

63. The method of claim 58, wherein said in situ hybridization stain is selected from the group consisting of fluorescent in situ hybridization (FISH) stain, radiolabeled in situ hybridization stain, Digoxigenin labeled in situ hybridization stain and biotinylated in situ hybridization stain.

64. The method of claim 58, wherein said DNA stain is a DNA-binding fluorescent dye.

65. The method of claim 55, wherein a first stain of said at least two stains is a morphological stain and a second stain of said at least two stains is selected from the group consisting of an immunological stain, an activity stain, an in situ hybridization stain, and a DNA stain.

66. The method of claim 55, wherein a first stain of said at least two stains is an immunological stain and a second stain of said at least two stains is selected from the group consisting of a morphological stain, an activity stain, an in situ hybridization stain and a DNA stain.

67. The method of claim 55, wherein a first stain of said at least two stains is an activity stain and a second stain of said at least two stains is selected from the group consisting of a morphological stain, an immunological stain, an in situ hybridization stain, and a DNA stain.

68. The method of claim 55, wherein a first stain of said at least two stains is a cytogenetical stain and a second stain of said at least two stains is selected from the group consisting of an immunological stain, an in situ hybridization stain, and a DNA stain.

69. The method of claim 55, wherein a first stain of said at least two stains is an in situ hybridization stain and a second stain of said at least two stains is a DNA stain.

70. The method of claim 55, wherein a first stain of said at least two stains is a DNA stain and a second stain of said at least two stains is an in situ hybridization stain.

71. The method of claim 55, wherein step (b) is effected using an automated cell imaging device capable of at least dual imaging.
